(19) United States
(12) Patent Application Publication
(10) Pub. No.: US 2013/0231590 A1
(43) Pub. Date: Sep. 5, 2013

(54) SYSTEM AND METHOD FOR ANALYZING GAIT AND RECOMMENDING FOOTWEAR WITH A MOBILE COMPUTER DEVICE

(71) Applicants: Justin Corbett, Kirkland, WA (US); David Hansen, Mercer Island, WA (US)

(72) Inventors: Justin Corbett, Kirkland, WA (US); David Hansen, Mercer Island, WA (US)

(73) Assignee: CORBETT ALLAN, INC., Kirkland, WA (US)

(21) Appl. No.: 13/411,595

(22) Filed: Mar. 4, 2012

(51) Int. Cl. A61B 5/11

(52) U.S. Cl. 600/595

(57) ABSTRACT

A mobile computer system and method for analyzing a runner's gait and selecting running shoes appropriate for the runner's gait. A mobile computer device with a video camera, user interface, and a processor coupled to a memory is presented to a user. A user records video of a runner's gait. A still frame that shows an anatomical joint in an evaluation position is selected. A simplified model of the anatomical joint is created. One or more shoes from a database of shoe profiles is selected according to the deviation of the simplified model from a physiological normal model. Consumer information regarding one or a plurality of the running shoes is displayed to the user. The user is enabled to purchase one or a plurality of the selected running shoes via e-commerce. Consumer and gait analysis information is shared with other computers via wireless or computer network.

### BACKGROUND

There is a standing industry and history of video analysis of human subjects running on treadmills in order to capture the moment of foot contact with the ground. Current systems utilize standalone desktop computer systems with specialized video software systems connected to dedicated video cameras through video capture cards, in connection with a treadmill station. Existing systems are expensive to purchase and assemble, and cumbersome to operate as they require trained technicians and dedicated space.

There exists a need for an integrated system for running analysis that is handheld, mobile, and that relies primarily upon a single electronic device. This system needs to operate without a trained gait analyst, to analyze a runner's gait and and recommend proper footwear.

### BRIEF SUMMARY OF THE INVENTION

The invention draws upon computer hardware improvements to bring to the shoe consumer an integrated system and method for analyzing their gait and recommending running shoes appropriate for that consumer's specific gait pattern. A mobile computer device with a video camera, user interface, and a processor coupled to a memory, wherein that processor is programmed to execute steps of the method described herein, is presented to a user. The method of analysis and recommendation is comprised of steps for recording video of a runner's gait, selecting a still frame of the video that shows an anatomical joint of the runner in an evaluation position, creating a simplified model of the anatomical joint, selecting one or a plurality of shoes from a database of running shoe profiles according to the deviation of the simplified model from a physiological normal model for the anatomical joint, and displaying to the user consumer information regarding one or a plurality of the running shoes. Alternative embodiments of the invention are additionally comprised of steps for enabling the user to purchase one or a plurality of the selected running shoes via e-commerce, and sharing consumer and gait analysis information with other computers via wireless or computer network.

### BRIEF DESCRIPTION OF THE DRAWINGS

FIG. 1 depicts an exemplary method for analyzing a runner's gait and recommending footwear with a mobile computer device; and

FIG. 2 depicts an exemplary screen shot of a video still frame with a simplified model of an anatomical joint and physiological reference points.

### DETAILED DESCRIPTION

An example embodiment of the present invention may be the following system and method for analyzing a runner's gait and recommending footwear with a mobile computer device.

A mobile computer system for analyzing a runner's gait may include a runner, a user of a mobile computer device, and a mobile computer device that is programmed to execute computer instructions for carrying out applicable method steps described herein. The value of the system is in its simplicity, mobility, reliance on commercially available hardware, and ease of use.

Several commercially available consumer products offer all of the necessary hardware components in the mobile computer device. An example of such a mobile computer device that was used in the development of the invention is the Apple iPad 2. Although an iPad 2 was used in the development of the invention, as used herein, a “mobile computer device” may be any single mobile computerized device having, in addition to other components, an integrated (i) video camera, (ii) processor, (iii) memory, (iv) user interface, and that is capable of configuration for receiving, storing and/or processing data for standalone application and/or over a networked medium or media, for example and without limitation, a laptop computer, tablet computer, personal data assistant (PDA), or mobile phone. Some embodiments of the invention also require that the mobile computer device have a wireless capability to connect to other computers over networks such as the mobile phone networks or the Internet.

Mobile computer devices described herein may include operatively associated computer-readable memory media such as memory for storing software applications and instructions used in obtaining, processing, storing and/or communicating data. It can be appreciated that such memory can be, in part, internal, external, remote or local with respect to its operatively associated computer or computer system. Memory may also include any means for storing software or other instructions including, for example and without limitation, a hard disk, an optical disk, DVD, compact disc, Blu-ray disc, memory stick, ROM (read only memory), RAM (random access memory), PROM (programmable ROM), EEPROM (extended erasable PROM), and/or other like computer-readable memory media.

In general, computer-readable memory media may include any memory medium capable of being a memory for electronic data representative of data or computer instructions stored, communicated, or processed in accordance with embodiments of the present invention. Where applicable, method steps described herein may be embodied or executed as computer instructions stored on a computer-readable memory medium or media.

Mobile computer devices described herein may include operatively associated digital image camera/video collection sensors for collection of video data. As used herein, a “video camera” may include any physical means for capturing a digital image or series of images including, for example and without limitation, a CMOS APS (complementary metal-oxide-semiconductor active pixel sensor) or a CCD (charge-coupled-device).

Mobile computer devices described herein may include an operatively associated user interface that serves to enable the user to interact with and control the mobile computer device. Many mobile computer devices such as the Apple iPad use a touchscreen as a user interface device, although many mobile computer devices such as laptops rely on a video screen operatively associated with a keyboard, mouse, or trackball to facilitate user interaction with and control of the mobile computer device. It can be also be appreciated that future developments, including those in gesture recognition, may additionally serve as a means for user interaction and control.

The mobile computer device described herein may use of an wireless interface that is operatively connected to an antenna for communicating data wirelessly with other computers or computerized databases via the Internet or other computer networks, using for example and without limitation, IEEE 802.11 protocols, 802.12.4 protocol, or other telecommunications protocols broadly characterized as 3G, 3G evolved technologies such as WiMax or LTE, 4G IMT-Advanced, or other protocol. Those skilled in the art of data networking will appreciate that many functional alternatives are possible for enabling the wireless communication between the mobile computer device and other computers or computerized databases. As used herein, the term “database” is accorded its ordinary meanings within the context of computer data and computer hardware systems.

A user of the mobile computer device generally interacts with the mobile computer device via the user interface to position the device correctly in relation to the runner and to begin and stop recording of video. The user may also be provided with video editing software for centering and cropping a video if such function is not automated by a software application.

The runner runs away from, towards, or across the field of view depending on what aspect of the runner's gait (i.e. the angle and position of a particular anatomical joint, discussed more fully infra) is being analyzed. Anatomical joint angle and position is best discerned when the two-dimensional plane captured in the video image is as close to perpendicular to the axis of movement of the anatomical joint being analyzed. For example, analysis of pronation or supination of the runner's ankle joint is most easily accomplished from the posterior view of a coronal anatomical plane, and analysis of the flexion and extension of the ankle or knee joints is most easily accomplished from either side of a sagittal anatomical plane. The mobile nature of the computer device allows for rapid switching between analyses.

Except where infeasible, the runner and the user may be the same person. As used herein, a “runner” may in fact be engaged in any type of bipedal motion, and does specifically include, without limitation, activities such as walking or sidestepping. Also, as used herein, “running shoe” should be interpreted broadly to include all types of footwear, and not just running-specific shoes of the preferred embodiment.

Computer instructions for carrying out applicable method steps described herein (herein described as a “software application”) were developed initially with Apple APIs (application programming interface) for use with Apple's iOS mobile OS (operating system), although it can be appreciated that any API may be used for development of similar computer instructions applications, including, but not limited to, those APIs for use with any version of the Android OS, Windows Mobile, PalmOS, Windows OS, Mac OS or other computer operating system.

In the preferred embodiment of the invention, the software application is capable of recognizing the shape of a runner's form and identifying position information regarding that runner's joint alignment in still frames of the captured video. Developed with the use of Apple APIs called the Core Image (CI) framework, stereotyped shape recognition can be built into the capability of the software application. Already built into iOS is a capability for automatic face detection, and body detection features are built into the features of the Mac OSX operating system. Using this CI API, or by employing similar methods, persons of ordinary skill in the art are capable of creating software that recognizes in still frames of captured video the basic shapes the human form such as the legs of the runner. For example, video still frame image may be processed and the angle of a joint such as the ankle or knee maybe be determined by differentiating the leg from the background (by converting the color composition of the still frame to sepia tones, increasing the level of contrast, converting to a binary black and white image) and simplifying the joint to three points that represent the anatomical joint angle and position (by determining the boundaries of the leg by mapping the silhouette edges, calculating a mean line between those edges, and simplifying as two lines between three points with one point at the joint angle vertex).

Similar image processing of video still frames and comparison of the resulting simplified joint models in sequence lends itself to a software application capable of discerning patterns of movement, useful for the identification of stereotyped positions of a runner's gait.

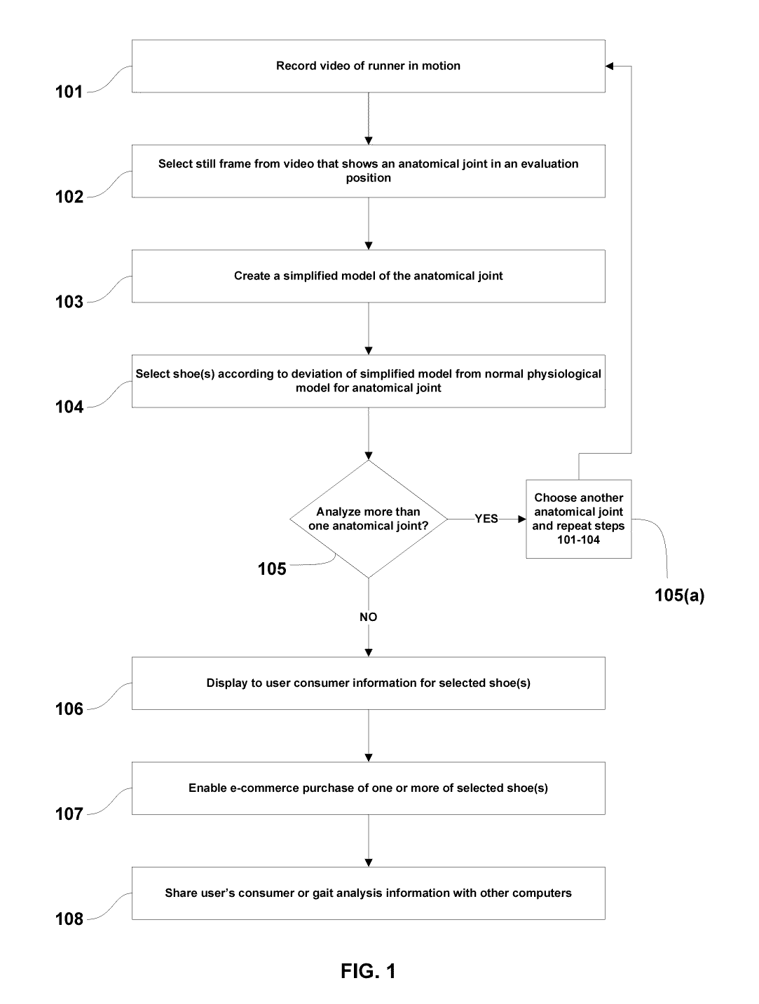

FIG. 1 depicts an exemplary process for analyzing a runner's gait and recommending appropriate footwear.

In block 101, a user records with the mobile computer device video of the runner in motion. A treadmill may be utilized, but is not necessary as it was with older systems, as software can allow for cropping and/or centering of the resulting video as a whole or as to individual still frames. The portable nature of the mobile computer device allows the user to take video of the runner from several perspectives without the significant effort involved in repositioning video hardware of former static systems.

In block 102, a still frame that shows a runner's form in an evaluation position is selected from the recorded video data. As used herein, the “evaluation position” means a general physiological moment and position in the cycle of a runner's gait that sets a standard reference point to compare to runner's gait to that of other runners or a physiological normal or control. When analyzing the pronation or supination of the ankle joint, one such suitable evaluation position is the moment of the foot's initial contact with the running surface and arrest of downward movement (the point of heelstrike). Contextual information regarding the horizon can also be gathered using image processing techniques or by the use of accelerometer/position sensing functionalities built into many commercially available mobile computer devices.

In an alternative embodiment of the invention, selection of the video still frame that shows the runner in an evaluation position is not accomplished directly by the software application, but rather is selected by presenting the user with video manipulation controls such as pause, rewind, advance, and frame by frame advance, and allowing the user to make a judgment about which individual frame best shows the runner's gait in an evaluation position.

As used herein, “anatomical joint” may mean any single human joint relevant to the analysis of a runners' gait and prescription of running shoes, such as the joints of the runner's feet, ankles, knees, or hips. It can be appreciated that each joint may have more than one axis of rotation or movement, and as such may yield more than one measurement useful for recommending a running shoe. For example, analysis of the ankle joint can yield measurements of overpronation/supination and of flexion/extension.

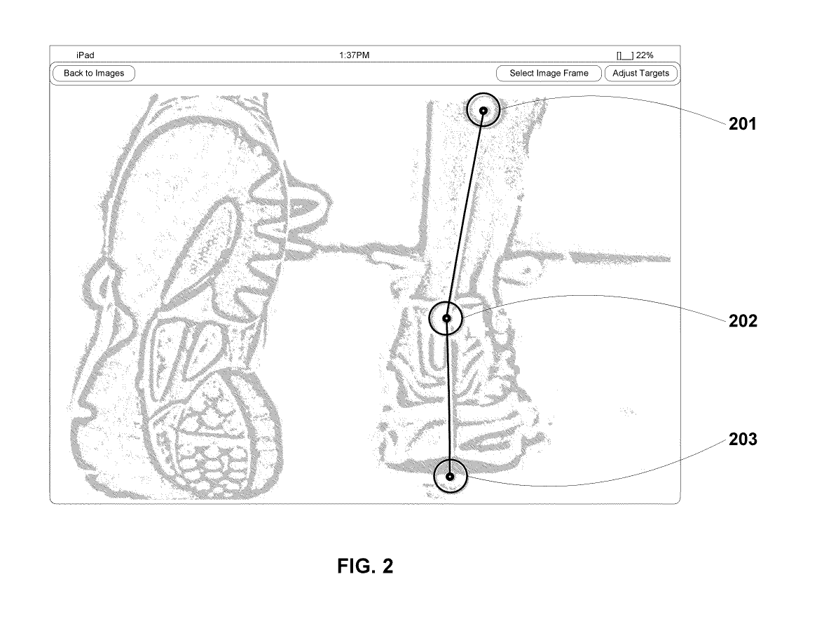

In block 103, a set of three reference points serving as the foundation of a simplified model of the anatomical joint being analyzed are selected on the still frame that shows the joint in the evaluation position, and FIG. 2 shows an exemplary screen shot of a video still frame with a simplified model of an anatomical joint and physiological reference points. These three reference points 201, 202, 203 define the approximations of the leg location and position: one reference point 202 representing the axis of movement of the anatomical joint, and two additional points 201, 203 distal along simplified medial lines representing the anatomical extensions beyond the anatomical joint. As used herein, a “simplified model” for an anatomical joint means the collection of data describing the position of the runner's leg, and may include position data for three reference points, the angles formed by these three points, a physiological descriptor for the angles formed, and in some cases contextual data regarding a horizon. For example, a simplified model of the ankle joint suitable for measuring ankle pronation or supination may include position data for one reference point 202 representing the axis of movement of the ankle joint, position data for two additional points 201 203 distal along simplified medial lines representing the superior and inferior anatomical extensions beyond the ankle joint, and information describing the angles formed at the axis reference point 202 as medial or lateral.

In an alternative embodiments of the invention, the user will either select placement for these three reference points manually, or may adjust placement of these points if the user judges the mobile computer system to have made inaccurate or improper placement.

In block 104, one or more running shoes are selected with the mobile computer device from a database of running shoe profiles according to the data of the simplified model. Data of the simplified model is compared with data that would be regarded as a physiological normal model, and values are assigned that describe the simplified model's variation from a physiological normal model. Such a physiological normal model may be derived from medical expertise, by statistical analysis of data developed from several iterations of the methods described herein, or by other methods.

The database of running shoe profiles associates running shoes with data ranges, wherein the data ranges represent a grouping of values describing variance in a runner's anatomy and/or gait characteristics, in values expressed by, relative to, or derived from the data of the simplified models. When data from the simplified model falls within the data range described by a particular shoe profile, that associated running shoe is selected. One or more running shoes may be selected. The groupings of values may be determined by statistical interpretation of standard deviation, measurement of joint angles by degrees or position, or by any other system of data measurement that allows for comparison and grouping in data ranges. For example, an ankle joint that shows in the simplified model an overpronation that deviates from a normal anatomical measurement by X standard deviations, and all running shoes having a profile with data ranges inclusive of X standard deviations of overpronation are selected.

Block 105 represents a user choice to conduct additional analysis of other anatomical joints of the same runner for use in finding running shoes selected according to the characteristics of more than one simplified model. Block 105(a) describes repetition of the method described in blocks 101-104 for different anatomical joints of the same runner allows for the narrowing of selections from the database of running shoe profiles and more developed recommendation for a running shoe. For example, an initial analysis of an ankle joint shows in its simplified model overpronation that deviates from a normal anatomical measurement by X standard deviations, and the user chooses to conduct a second analysis of the a knee joint that that shows in its simplified model hyperextension that deviates from a normal anatomical measurement by Y standard deviations, only those running shoes having profiles with data ranges inclusive of X standard deviations of overpronation and Y standard deviations of hyperextension would be selected.

In block 106, consumer information regarding one or more selected running shoes is presented to the user with the mobile computer device. As used herein, “consumer information” means information relevant to a consumer's decision to purchase a product or service, and may include price, images, descriptions, and other information common retailing and/or e-commerce retailing.

In block 107, alternative embodiments of the invention additionally present the user with a mechanism for purchasing one or several of the selected running shoes online with the mobile computer device. Many commercially available mechanisms for purchasing goods or services with a mobile computer device exist, including but not limited to the presentation of hyperlinks to products available from online e-commerce retailers or integration of computer instructions for an e-commerce cart into the software application.

In block 108, resulting consumer and gait analysis information gathered with the mobile computer device may be shared with other computers, via wireless or computer networks such as a mobile phone network or the Internet. As this system is intended to analyze the gaits of 20,000+runners per year, the consumer and scientific information contained in each analysis is of significant value when shared. Such information may be used to for advertising, product development, further development of running shoe profile data, or to allow runners to share their experience via social networking websites.

It is to be understood that the figures and descriptions of the present invention have been simplified to illustrate elements that are relevant for a clear understanding of the present invention, while eliminating, for purposes of clarity, other elements. Those of ordinary skill in the art will recognize, however, that these and other elements may be desirable. However, because such elements are well known in the art, and because they do not facilitate a better understanding of the present invention, a discussion of such elements is not provided herein. It should be appreciated that the figures are presented for illustrative purposes and not as construction drawings. Omitted details and modifications or alternative embodiments are within the purview of persons of ordinary skill in the art.

It can be appreciated that, in certain aspects of the present invention, a single component may be replaced by multiple components, and multiple components may be replaced by a single component, to provide an element or structure or to perform a given function or functions. Except where such substitution would not be operative to practice certain embodiments of the present invention, such substitution is considered within the scope of the present invention.

The examples presented herein are intended to illustrate potential and specific implementations of the present invention. It can be appreciated that the examples are intended primarily for purposes of illustration of the invention for those skilled in the art. The diagrams depicted herein are provided by way of example. There may be variations to these diagrams or the operations described herein without departing from the spirit of the invention. For instance, in certain cases, method steps or operations may be performed or executed in differing order, or operations may be added, deleted or modified.

Furthermore, whereas particular embodiments of the invention have been described herein for the purpose of illustrating the invention and not for the purpose of limiting the same, it will be appreciated by those of ordinary skill in the art that numerous variations of the details, materials and arrangement of elements, steps, structures, and/or parts may be made within the principle and scope of the invention without departing from the invention as described in the following claims.

What is claimed is:

1. A method of analyzing a runner's gait and selecting running shoes appropriate for the runner's gait using a mobile computer system, comprising:
recording with the mobile computer system a video of a runner's gait;
selecting with the mobile computer system a still frame of from the video that shows an anatomical joint in an evaluation position;
creating with the mobile computer system a simplified model of the anatomical joint;
selecting with the mobile computer system one or a plurality of running shoes from a database of running shoe profiles according to the deviation of the simplified model from a physiological normal model for the anatomical joint; and
displaying to the user with the mobile computer system consumer information regarding one or a plurality of the selected running shoes.

2. The method of claim 1, wherein the still frame showing the anatomical joint in the evaluation position is selected by the user.

3. The method of claim 1, wherein reference points of the simplified model are selected or adjusted by the user.

4. The method of claim 1, wherein the anatomical joint in the evaluation position and the reference points of the simplified model are selected or adjusted by the user.

5. The method of any one of claims 1-4, further comprising repetition of the claimed steps for recording a video, selecting a still frame, creating a simplified model, and selecting running shoes, wherein one or a plurality of other anatomical joints of the runner are analyzed, and wherein only consumer information for running shoes commonly selected for all anatomical joints analyzed are displayed.

6. The method of any one of claims 1-4, further comprising a step for enabling the user to purchase one or a plurality of the selected running shoes via e-commerce.

7. The method of claim 5, further comprising a step for enabling the user to purchase one or a plurality of the selected running shoes via e-commerce.

8. The method of claim 6, further comprising a step for using the mobile computer device to share consumer and gait analysis information with other computers via wireless or computer network.

9. The method of claim 7, further comprising a step for using the mobile computer device to share consumer and gait analysis information with other computers via wireless or computer network.

10. An mobile computer system for analyzing a runner's gait and selecting running shoes appropriate for the runner's gait comprising:
a video camera;
a user interface;
and a processor coupled to a memory,
wherein the processor is programmed to provide analysis of a runner's gait and select appropriate running shoes by:
recording a video of a runner's gait,
selecting a still frame of from the video that shows an anatomical joint in an evaluation position,
creating a simplified model of the anatomical joint,
selecting one or a plurality of running shoes from a database of running shoe profiles according to the deviation of the simplified model from a physiological normal model for the anatomical joint, and
displaying to the user consumer information regarding one or a plurality of the selected running shoes.

11. The system of claim 10, additionally comprising a means for communicating data wirelessly with other computers or computerized databases via the Internet or other computer networks and wherein the processor is additionally programmed to provide an e-commerce means for purchasing one or a plurality of the selected running shoes.

12. The system of either claim 10 or claim 11, wherein the user interface is a touchscreen display.

12. The system of claim 12, wherein the processor is additionally programed to share consumer and gait analysis information with other computers via wireless or computer network.
